(19) United States
(12) Patent Application Publication
(10) Pub. No.: US 2017/0362566 A1
(43) Pub. Date: Dec. 21, 2017

(54) Polymers for Inducing 3D Spheroid Formation of Biological Cells

(71) Applicants: John Solomon GARNER, West Lafayette, IN (US); Sarah Michelle SKIDMORE, Lafayette, IN (US); Justin Charles HADAR, Lafayette, IN (US); Burnsoo HAN, West Lafayette, IN (US); Kinam PARK, West Lafayette, IN (US); AKINA, INC., West Lafaytte, IN (US)

(72) Inventors: JOHN SOLOMON GARNER, West Lafayette, IN (US); SARAH MICHELLE SKIDMORE, Lafayette, IN (US); JUSTIN CHARLES HADAR, West Lafayette, IN (US); Burnsoo HAN, West Lafayette, IN (US); KINAM PARK, West Lafette, IN (US)

(21) Appl. No.: 15/533,027

(22) PCT Filed: Dec. 7, 2015
(86) PCT No.: PCT/US15/64271

§ 371 Date: Jun. 4, 2017

Related U.S. Application Data
(60) Provisional application No. 62/088,431, filed on Dec. 5, 2014.

(51) Int. Cl. C12N 5/00; C08F 126/06; C08B 3/10; C08G 18/48; C08G 18/76; C12N 5/09; C08F 220/54

(52) U.S. Cl. CPC C12N 5/0062; C12N 5/0693; C08F 126/06; C08F 220/54; C08G 18/4825; C08G 18/7671; C08B 3/10; C08G 2220/00; C12N 2513/00; C12N 2533/30; C12N 2533/78; C12N 2537/00; C12N 2539/00

(57) ABSTRACT

The present invention provides the use of selected thermogelling polymers for the purpose of growing tumor spheroids. The invention provides a thermogelling platform comprising a synthetic polymer which, when seeded with cancer cells, induces the cells to grow into a natural spheroidal pattern forming a tumor spheroid. After accomplishing this in about 3-10 days, the gel washes away, leaving behind the spheroids.

### CROSS REFERENCE

This application is the national stage of international application number PCT/US2015/064271, filed Dec. 7, 2015, which claimed benefit of U.S. provisional application No. 61/739,072, filed Dec. 5, 2014, the subject matter of each of the above referenced disclosures is expressly incorporated by reference herein.

### FIELD OF INVENTION

The present invention relates to the application of synthetic thermogelling polymers to the growth of spheroidally-formed biological cells. While cells grown in a 2D monolayer or other format lack physiological relevancy, spheroids are more representative of tumor formation as they exhibit increased cell survival, relevant morphology, and hypoxic core which is seen in native tumors but not in 2D or other tumor models. In order to assay potential treatments and for other research applications, there is a need for a 3D cell model, such as a spheroid, which accurately represents in-vivo conditions. This invention allows for the convenient growth of cell spheroids or other three-dimensional structures by co-incubation.

### BACKGROUND OF THE INVENTION

Cells are cultured for a variety of reasons, including drug screening in cancer research, tissue engineering, tumor modeling, gene function analysis, and cell-cell interaction analysis (R. Z. Lin et al., “Recent advances in three-dimensional multicellular spheroid culture for biomedical research”, Biotechnology Journal 3 (2008) pp. 1172-1184). Two-dimensional (2D) cell culture was initially developed in 1908 (Harrison, R. G. et al. “Observations of the living developing nerve fiber”. Anat. Rec. 1, (1907) p. 116-128). While 2D cell culture has enjoyed a long history of use in the laboratory, it has been noted by researchers that cells cultured in a three dimensional (3D) environment (spheroid) provide more clinically relevant data than cells cultured in a monolayer. When cells are grown into a spheroid they are able to have cell-cell interactions as well as cell-extracellular matrix (ECM) interactions. These interactions more closely mimic the in vivo environment and can lead to more accurate data generation, particularly when drug screening tests are performed (S. Breslin, et. al. “Three-dimensional cell culture: the missing link in drug discovery”, Drug Discovery Today 18 (2013) pp. 240-249).

Spheroids have been used by researchers to study multicellular resistance to drugs (Desoize, B. et al., “Cell culture as spheroids: an approach to multicellular resistance.” Anticancer Res. 18 (1998), pp. 4147-4158 and Djordjevic, B., Lange, C. S., “Measurement of sensitivity to adriamycin in hybrid spheroids.” Cancer Invest. 9 (1991), pp. 505-512). Spheroids are utilized in tumor modeling to determine signaling pathways and cell interactions to provide insight into drug treatment options, as well as drug development (Dardousis, K. et al., “Identification of differentially expressed genes involved in the formation of multicellular tumor spheroids by HT-29 colon carcinoma cells.” Mol. Ther. (2007) 15, pp. 94-102; Ghosh, S. et al., “Use of multicellular tumor spheroids to dissect endothelial cell-tumor cell interactions: a role for T-cadherin in tumor angiogenesis”. FEBS Lett. 581 (2007), pp. 4523-4528; Feder-Mengus, et al., “Multiple mechanisms underlie defective recognition of melanoma cells cultured in three-dimensional architectures by antigen-specific cytotoxic T lymphocytes.” Br. J. Cancer 96 (2007), pp. 1072-1082; and Durand, R. E., “Slow penetration of anthracyclines into spheroids and tumors: a therapeutic advantage?” Cancer Chemother. Pharmacol. 26 (1990), pp. 198-204). Spheroids are used extensively in drug screening platforms (Bartholoma, P., et al., “A more aggressive breast cancer spheroid model coupled to an electronic capillary sensor system for a high-content screening of cytotoxic agents in cancer therapy: 3-dimensional in vitro tumor spheroids as a screening model.” J. Biomol. Screen. 10 (2005), pp. 705-714; Friedrich, J., et al., “Spheroid-based drug screen: considerations and practical approach.” Nature Protoc. 4 (2009), pp. 309-324; Kunz-Schughart L A, et al., “The use of 3-D cultures for high-throughput screening: the multicellular spheroid model.” J Biomol Screen 9 (2004) pp. 273-285.). Spheroids have provided a useful alternative to mouse models in cancer research (Dubessy, C., et al., “Spheroids in radiobiology and photodynamic therapy.” Crit. Rev. Oncol. Hematol. 36 (2000), pp. 179-192). Spheroids are a possible material for use in tissue engineering to reconstruct organs and whole tissues (Lin R Z, et al., “Magnetic reconstruction of three-dimensional tissues from multicellular spheroids.” Tissue Eng Part C Methods 14 (2008) pp. 197-205; Marga F, et al., “Developmental biology and tissue engineering.” Birth Defects Res C Embryo Today 81 (2007) pp. 320-328; Steer D L, Nigam S K. “Developmental approaches to kidney tissue engineering.” Am J Physiol Renal Physiol 286 (2004) pp. F1-F7).

Developing a 3D culture method has not always been a simple and inexpensive process. Currently, there are several methods to induce cells to form a spheroid. One method is known as the “forced-floating” method. The surface upon which the cells grow is modified in a way that the cells are unable to attach to the surface. The cells are then forced to float, which induces cell-cell interactions leading to spheroid formation. (Ivascu, A. and Kubbies, M. “Rapid generation of single-tumor spheroids for high-throughput cell function and toxicity analysis.” J. Biomol. Screen. 11 (2006), pp. 922-932; Friedrich, J. et al. “Spheroid-based drug screen: considerations and practical approach.” Nat. Protoc. 4 (2009), pp. 309-324; Li, Q. et al. “3D models of epithelial-mesenchymal transition in breast cancer metastasis.” J. Biomol. Screen. 16 (2011), p. 141-154). The pre-coating of the plates needed to develop spheroids using the forced-floating method can be time-consuming and labor intensive. Pre-coated plates are available to purchase from suppliers such as Sumitomo Bakelite (www.sumibe.co.jp/english/product/s-bio/cell-culture/primesurface-96u/index.html), EMD Millipore (www.emdmillipore.com/US/en/life-science-research/cell-culture-systems/cell-growth/aEGb.qB.Cn4AAAE_kBd3.Mm_,nav) and Happy Cell (www.happy-cell.com/happy-shop/low-attachment-plates/). However, purchasing pre-coated plates adds additional cost to the researcher's projects.

Another method of inducing spheroid formation is called the “hanging drop” method. In this method, a cell suspension is seeded on a plate, which is then inverted and incubated. The cell suspension relies on surface tension to adhere to the plate while inverted. The cells form spheroids after sinking to the bottom of the drop upon inversion and culturing. (Kelm, J. M. et al. “Method for generation of homogeneous multicellular tumor spheroids applicable to a wide variety of cell types.” Biotechnol. Bioeng. 83 (2003), pp. 173-180). While this method produces uniform spheroids, media change is very difficult and the volume of the drop containing cells is limited (Kurosawa, H. “Methods for inducing embryoid body formation: in vitro differentiation system of embryonic stem cells.” J. Biosci. Bioeng. 103 (2007), pp. 389-398). “Hanging drop” plates are commercially available from several suppliers including 3D Biomatrix (www.3dbiomatrix.com) and InSphero (www.insphero.com).

Yet another method for producing cell spheroids is an agitation-based procedure. In this method, either the cells are stirred (Kim, J. B. “Three-dimensional tissue culture models in cancer biology.” Semin. Cancer Biol. 15 (2005), pp. 365-377) or the container the cells are in is rotated (Barrila, J. et al. “Organotypic 3D cell culture models: using the rotating wall vessel to study host pathogen interactions.” Nat. Rev. Microbiol. 8 (2010), pp. 791-801). The rotation of the cells promotes cell-cell interactions and inhibits cell adherence to the container walls. Some drawbacks of this method include large volume of media required, possible detrimental effect on cell physiology from rotation and limited control over spheroid size (Lin, R. Z. and Chang, H. Y. “Recent advances in three-dimensional multicellular spheroid culture for biomedical research.” Biotechnology Journal, 3 (2008), pp. 1172-1184). Several companies have commercial agitators available, including Wheaton (www.wheatonsci.com), Corning (www.corning.com) and Synthecon (www.synthecon.com).

Another method for producing tumor spheroids involves the use of a matrix for spheroid development. The cells are either seeded on, or in, a matrix. This matrix is a commercially purchased extra-cellular matrix that consists of biological material similar to what the cell would be exposed to in-vivo. The cost of purchasing this matrix may be prohibitive to most researchers and the composition of the matrix is not always consistent from batch to batch since it is a biological product. (Sodunke, T. R. et al. “Micropatterns of Matrigel for three-dimensional epithelial cultures.” Biomaterials 28 (2007), pp. 4006-4016). Several ECM options are commercially available from companies such as BD Biosciences (www.bdbiosciences.com/ca/cellculture/mimetic/index.jsp), Amsbio (www.amsbio.com/animal-free-chemically-defined-collagen-laminin-fibronectin-vitronectin-ECM.aspx), Sigma-Aldrich (www.sigmaaldrich.com/catalog/product/sigma/e1270?lang=en&region=US), Millipore (www.emdmillipore.com/US/en/product/ECM-Proteins-and-Coated-Plates,MM_NF-C77812) and Invitrogen (www.lifetechnologies.com/us/en/home/life-science/cell-culture/3d-cell-culture.html?cid=fl-3d-cellculture).

An additional method for producing spheroids involves the use of prefabricated scaffolds. Cells are seeded into the scaffold, where they attach to the fibers of the scaffold, and then grow into 3D structures. (Sourla, A. et al. “Three-dimensional type I collagen gel system containing MG-63 osteoblasts-like cells as a model for studying local bone reaction caused by metastatic cancer cells.” Anticancer Res. 16 (1996), pp. 2773-2780). Some difficulties with this method include the mechanical strength of the scaffold, cost and inability to remove the cells easily from the scaffold. Various scaffold types are commercially available from companies such as 3D Biomatrix, Sigma-Aldrich, Solohill (www.pall.com/main/biopharmaceuticals/solohill-microcarriers.page), and Amsbio (www.amsbio.com).

As discussed above, the various methods of cell spheroid formation present numerous challenges to the researcher. The present invention eliminates these challenges with a novel platform for spheroid growth. This method is simple, inexpensive, and does not require extra equipment for spheroid development.

### SUMMARY OF THE INVENTION

The present invention provides the use of selected thermogelling polymers for the purpose of growing biological cells as spheroids. While the invention encompasses all biological cell types, tumor cells are preferred. For example, the invention provides a thermogelling platform comprising a synthetic polymer which, when seeded with cancer cells, induces the cells to grow into a natural spheroidal pattern forming a tumor spheroid. After accomplishing this in about 3-10 days, the gel washes away, leaving behind the spheroids.

In a 1^{st} aspect, the present invention provides a platform for inducing 3D spheroid formation of biological cells, comprising: a thermogelling polymer without amino acids.

In a 1^{st} embodiment, the thermogelling polymer is chemically synthetic or semi-synthetic.

In a 2^{nd} embodiment, the thermogelling polymer is a stearate-modified methyl cellulose.

In a 3^{rd} embodiment, the thermogelling polymer is poloxamer 407 linked by polyurethane linkages.

In a preferred embodiment, the polyurethane linkages are generated utilizing hexamethyldiisocyanate.

In another preferred embodiment, the polyurethane linkages are generated utilizing methylene diphenyldiisocyanate.

In another preferred embodiment, the polyurethane linkages are generated utilizing toluene diisocyanate.

In a 4^{th} embodiment, the thermogelling polymer is a hydrophobically-modified cellulose derivative.

In a 5^{th} embodiment, the thermogelling polymer is a cellulose derivative modified by chemical conjugation of stearic acid units.

In a 6^{th} embodiment, the thermogelling polymer is a cellulose derivative modified by chemical conjugation of methyl units.

In a 7^{th} embodiment, the thermogelling polymer is a cellulose derivative modified by chemical conjugation of hydroxypropyl units.

In an 8^{th} embodiment, the thermogelling polymer is a cellulose derivative modified by chemical conjugation of ethyl units.

In a 9^{th} embodiment, the thermogelling polymer is a cellulose derivative modified by chemical conjugation of propyl units.

In a 10^{th} embodiment, the thermogelling polymer is a cellulose derivative modified by chemical conjugation of hydroxyethyl units.

In an 11^{th} embodiment, the thermogelling polymer is a cellulose derivative modified by chemical conjugation of carboxymethyl units.

In a 12^{th} embodiment, the platform further comprises a cell growth medium in the range of about 1 to about 30% w/v.

In a preferred embodiment, the cell growth medium is in the range of about 5 to about 20% w/v.

In a 13^{th} embodiment, the biological cells are tumor cells.

In a 14^{th} embodiment, the biological cells are benign tumor cells.

In a 15^{th} embodiment, the biological cells are malignant tumor cells.

In a 16^{th} embodiment, the biological cells are cancer cells.

In a 17^{th} embodiment, the biological cells are breast cancer cells.

In an 18^{th} embodiment, the spheroids have diameters of about 10 μm to about 1000 μm.

In a 19^{th} embodiment, the spheroids have diameters of about 10 μm to about 500 μm.

In a 20^{th} embodiment, the spheroids have diameters of about 20 μm to about 200 μm.

In a 2^{nd} aspect, the present invention provides a method for inducing 3D spheroid formation of biological cells on a thermogelling polymer without amino acids, comprising the step of: combining the thermogelling polymer with a growth medium and the biological cells.

In a 1^{st} embodiment, the thermogelling polymer is chemically synthetic or semi-synthetic.

In a 2^{nd} embodiment, the thermogelling polymer is a stearate-modified methyl cellulose.

In a 3^{rd} embodiment, the thermogelling polymer is poloxamer 407 linked by polyurethane linkages.

In a preferred embodiment, the polyurethane linkages are generated utilizing hexamethyldiisocyanate.

In another preferred embodiment, the polyurethane linkages are generated utilizing methylene diphenyldiisocyanate.

In another preferred embodiment, the polyurethane linkages are generated utilizing toluene diisocyanate.

In a 4^{th} embodiment, the thermogelling polymer is a hydrophobically-modified cellulose derivative.

In a 5^{th} embodiment, the thermogelling polymer is a cellulose derivative modified by chemical conjugation of stearic acid units.

In a 6^{th} embodiment, the thermogelling polymer is a cellulose derivative modified by chemical conjugation of methyl units.

In a 7^{th} embodiment, the thermogelling polymer is a cellulose derivative modified by chemical conjugation of hydroxypropyl units.

In an 8^{th} embodiment, the thermogelling polymer is a cellulose derivative modified by chemical conjugation of ethyl units.

In a 9^{th} embodiment, the thermogelling polymer is a cellulose derivative modified by chemical conjugation of propyl units.

In a 10^{th} embodiment, the thermogelling polymer is a cellulose derivative modified by chemical conjugation of hydroxyethyl units.

In an 11^{th} embodiment, the thermogelling polymer is a cellulose derivative modified by chemical conjugation of carboxymethyl units.

In a 12^{th} embodiment, the cell growth medium is in the range of about 1 to about 30% w/v.

In a preferred embodiment, the cell growth medium is in the range of about 5 to about 20% w/v.

In a 13^{th} embodiment, the biological cells are tumor cells.

In a 14^{th} embodiment, the biological cells are benign tumor cells.

In a 15^{th} embodiment, the biological cells are malignant tumor cells.

In a 16^{th} embodiment, the biological cells are cancer cells.

In a 17^{th} embodiment, the biological cells are breast cancer cells.

In an 18^{th} embodiment, the spheroids have diameters of about 10 μm to about 1000 μm.

In a 19^{th} embodiment, the spheroids have diameters of about 10 μm to about 500 μm.

In a 20^{th} embodiment, the spheroids have diameters of about 20 μm to about 200 μm.

In a 21^{th} embodiment, the method further comprises the step of: incubating the biological cells with the thermogelling polymer and growth medium at appropriate conditions to support biological growth.

In a 22^{th} embodiment, the method further comprises the step of: harvesting the 3D spheroid formations of biological cells from the thermogelling polymer and growth medium.

It will be appreciated that all allowable combinations of the above aspects/embodiments, as well as other aspects/embodiments described elsewhere herein, are contemplated as further aspects/embodiment of the invention.

### BRIEF DESCRIPTION OF THE DRAWINGS

It will be appreciated that the following FIGURE relate to a particular embodiment of the invention discussed below. The FIGURE is not intended to limit the scope of the invention.

FIG. 1: MCF-7 cells growing in AO25 after one day of incubation.

### DETAILED DESCRIPTION OF THE INVENTION

### Definitions

As used herein,

The term “biological cell,” unless modified, refers to any biological cell.

Preferred biological cells include tumor and cancer cells.

The term “synthetic polymer” refers to a chemical substance, not derived from natural origins, consisting of repeating units linked together with covalent bonds.

The term “semi-synthetic polymer” refers to a polymer prepared by chemical synthesis starting from natural materials.

The term “biocompatible” refers to a material property which allows for the growth and/or proliferation of biological cells in its presence.

The term “growth medium” refers to a liquid or gel which supports the growth of microorganisms or biological cells. For example, in some embodiments, the medium contains water and nutrients.

The term “thermogel” refers to any polymeric material that, upon dissolution in water, possesses the property to change solution rheological properties upon change in solution temperature.

The invention relates to the use of a selected series of synthetic thermogel polymers for the purpose of growing cancer cells into spheroids. When dissolved in water at a gellable concentration (e.g., 1% to 50% w/v, preferably 5% w/v to 20% w/v at 5 C), the thermogel should have a viscosity of less than 100 Pa·s (pascal seconds), preferably less than 50 Pa·s, more preferably less than 10 Pa·s. In fact, as there is no lower limit, a viscosity of 0 is optimal. Unlike other growth media, these synthetic polymers are not derived from biological sources, and as such are lower in cost and higher in reproducibility than other materials used for this application. The synthetic pathways for these thermogels are described in examples below.

### Abbreviations

The following abbreviations are used throughout this document:

Abbreviations

| | |
|---|---|
| AA: | Acetic acid |
| AAc: | Acrylic acid |
| AAm: | Acrylamide |
| ACN: | Acetonitrile |
| ACE: | Acetone |
| AIBN: | 2,2′-azobisisobutyrylnitrile |
| Alginate: | Sodium salt of alginic acid |
| APS: | Ammonium persulfate |
| BIS: | N,N′-methylenebisacrylamide |
| BPO: | Benzoyl peroxide |
| DW: | Distilled water |
| DMSO: | Dimethyl sulfoxide |
| DCM: | Dichloromethane |
| EBA: | N, N′-ethylenebisacrylamide |
| EG-DA: | Ethylene glycol diacrylate |
| HEA: | Hydroxyethyl acrylate |
| HEMA: | Hydroxyethyl methacrylate |
| Hexet: | 80:20 Hexane:Ethanol (v:v) |
| MPEG: | Monomethoxy poly(ethylene |
| | glycol) |
| NIPAM: | N-isopropyl acrylamide |
| PAA: | Poly(acrylic acid) |
| PAAm: | Polyacrylamide |
| PEG: | Poly(ethylene glycol) |
| PVOH: | Poly(vinyl alcohol) |
| TEMED: | N,N,N′,N′- |
| | tetramethylethylenediamine |
| Sn(OCT): | Stanneous Octoate |
| F127: | Poloxamer 407 |
| HMDI: | Hexamethyldiisocyanate |
| MDI: | Methylenediphenyldiisocyanate |
| AO14: | Poly(N-isopropylacrylamide-co- |
| | acrylic acid) |
| AO20: | Poly(F127)-urethane |
| AO25: | Stearic acid modified methyl |
| | cellulose |
| AO26: | Poly(Poloxamer |
| | 407)methylenediphenyldiisocianate |
| AO31: | Poly(vinylcaprolactam) |

### Scale of Cell Aggregation

The following scale is used throughout this document:

| Level of | |
|---|---|
| Aggregation | Definition |
| 0 | Discrete spheroid growth, practically no |
| | aggregation (<5% aggregated) |
| 1 | Predominately discrete spheroid growth, some |
| | spheroid aggregates (approximately 5-30% |
| | aggregated) |
| 2 | Fairly even mixture of discrete spheroid |
| | growth and spheroid aggregation |
| | (approximately 30-60% aggregated) |
| 3 | Predominately spheroid aggregates, some |
| | discrete spheroid growth (approximately 60- |
| | 95% aggregated) |
| 4 | Practically complete aggregated growth/ |
| | sheet like cell structures (>95% aggregated) |

### I. Syntheses

### Example 1: AO14 Poly(N-Isopropylacrylamide-Co-Acrylic Acid)

This copolymer is synthesized by charging into a 1000 ml 1 neck round-bottom-flask 75 grams of NIPAM, 5 ml of AAc, 240 ml of DW, 240 ml ACN, 0.4 ml TEMED, and 0.4 g APS. The solution is sparged with nitrogen gas and gentle warming at 60° C. to initiate polymerization. Post reaction, the polymer solution is dissolved in a mixture of DCM:ACE (1:1 v:v), passed through a filter, and precipitated in hexet. Obtained solid is dried under deep vacuum at room temperature.

### Example 2: AO20 Poly(F127-Urethane) (˜20 Units)

Into a 1 L RBF is put 100 g of F127 plus 4 grams mPEG (5000 Da). This is dried at room temperature under deep vacuum to remove surface water. These are then dissolved in anhydrous DCM and 10 ml of 10% w/v Sn(Oct)/toluene is added along with 1.35 ml of HMDI. The solution is sealed and heated at 60° C. overnight under reflux with a desiccant trap. Polymer is dissolved in dichloromethane, passed through a filter, and precipitated in hexet. Obtained solid is dried under deep vacuum at room temperature.

### Example 3: AO25 Stearic Acid Modified Methylcellulose

Into a 1000 ml flask 31.4 g of methylcellulose (Mn 14,000, 1.6-1.9 mol methoxy per mol cellulose, 27.5-31.5% wt % methoxy, 53-59 dynes/cm surface tension (25 C, 0.05%, 15 cP, gel point 50-55° C.) is added along with anhydrous ACN plus 2.2 ml triethylamine. This is stirred and heated to 90° C. to dissolve. To this solution 2.52 g of stearyloyl chloride is added along with 100 ml of DMSO, and the solution is stirred at room temperature for 3 days. Subsequently, this solution has its volume reduced to roughly ½-¾ of current volume under vacuum and then is precipitated in ethanol. Obtained solid is washed with ethanol and dried under deep vacuum at room temperature.

### Example 4: AO26 Poly(Poloxamer 407-Methylenediphenyldiisocianate)

Into a round-bottom-flask is placed 100 g F127, 0.8 g mPEG (5000 Da) and heated to 125° C. while under deep vacuum to dehydrate for 1 hour. Subsequently, 3 g of MDI is melted at 80° C. and combined with pre-heated F127/mPEG 5000 mixture. This is stirred rapidly, then allowed to cool to room temperature. After cooling, polymer is dissolved in DCM, filtered, and precipitated in hexet. Obtained solid is dried under deep vacuum at room temperature.

### Example 5: AO31 Poly(Vinylcaprolactam)

Into a 1000 mL, 2 neck round bottom flask 2.5 ml of 4% (w/v) AIBN/DMSO is added along with 50 g vinylcaprolactam. This is then dissolved with ˜300-500 ml acetonitrile. The solution is sparged for 30 minutes to remove oxygen, sealed off, and heated to 70° C. overnight to react. Subsequently, this polymer is precipitated in hexet, and the obtained solids are dried under deep vacuum at room temperature.

Characterization:

The hydrogel Examples described above are characterized by the following methods:

Rheological Characterization

Rheology was performed on a TA instruments AR550 equipped with a temperature controlled peltier plate. The geometry utilized is a 60 mm 2° cone with a 62 μm truncated tip. The peltier plate has a heatsink provided by a Neslab RTE10 refrigerated circulator set to pump solution through the peltier plate at 23° C. The air bearing is supported by 35 psi of compressed air. The instrument is operated by an Inspiron 530s Dell computer utilizing AR instrument control software. Data analysis is provided on same computer using TA universal data analysis software. Prior to initiating runs the instrument geometry was mapped utilizing AR instrument control software using settings of standard mapping speed at 3 iterations. Platen was set to an initial temperature of 5° C. Subsequently the gap was zero using software provided feature and gap was set to 100 μm for sample loading. Each polymer was dissolved in cold water as detailed in the examples and the generated solution was stored in an insulated container along with an icepack prior to testing.

Each sample (2 ml) was injected into the gap set to 100 μm and the gap was reset to 62 μm for the run. For the run, each sample was initially equilibrated at 5° C. for 1 min prior to testing. At 5° C., an applied shear rate of 0.1 sec⁻¹ was applied and the sample and the resultant viscosity was sampled at 5 second intervals. After this, the sample temperature was increased stepwise from 5° C. to 45° C. at 2.5° C. increments with 3 minutes of equilibration time at each increment. At each increment, 0.1% strain was applied with an oscillating frequency of 6.283 rad/sec with a conditioning time of 3 seconds and a sampling time of 3 seconds. After measuring at the final 45° C. temperature, the sample was returned to 5° C. The cone was raised and the cone/platen cleaned off prior to injecting the next sample. The viscosity of each sample at 5° C. was calculated from the initial shear rate run by averaging the viscosity values of each time interval. This value is listed in results section for each example.

Inherent Viscosity

For selected polymers in which gel permeation chromatography was not possible, inherent viscosity was performed in order to obtain molecular weight information. Each sample was prepared by dissolving at indicated concentration (typically 1-2% w/v (1-2 g/dL)) in indicated solvent as detailed in each example. Each sample was dissolved at room temperature. Samples were tested in a Cannon mini-viscometer (C286) in a water bath set to the indicated temperature. The test result (in seconds) was multiplied by the manufacturer provided viscometer constant (0.004039 cST/s) to obtain sample viscosity. The relative viscosity (Vᵣₑₗ) was obtained by dividing the sample viscosity by that of literature reported value for pure solvent. The inherent viscosity (IV) was obtained (in dL/g) by the following equation where “C” is the polymer concentration:

IV (dL/g)=ln(Vᵣₑₗ)/(C g/dL)

Testing was performed in triplicate and 3 sample results were averaged together for each example unless otherwise specified.

Gel Permeation Chromatography (GPC)

Select polymers were tested by GPC. Each sample was dissolved in 0.2 um filtered chromatography grade dichloromethane (DCM) (Mallinckrodt, Chromasolv). After dissolution, the sample was passed through a 0.45 μm PVDF filter to remove any particulates, and placed directly into a septa capped 2 ml HPLC vial. Gel-permeation chromatography was performed using a Varian prostar system equipped with a model 210 isocratic pump, a model 410 auto-sampler, and a model 335 photodiode array (PDA) detector. Elution was done with 1 ml/min flow of DCM across three columns in sequence containing a combination of sized phenogel and Resipore (Agilent, mixed bed) columns. Unless otherwise specified, absorbance of the sample was taken at 237 nm. Control was performed using Galaxie software package. Calibration of the system was performed using Agilent PS2 © polystyrene standard series “A” and “B” per MFG instructions. Calibration was performed in a “book-end” format with standards run before and after the sample runs and their retention times averaged to generate the calibration curve.

Hydrogen-Nuclear Magnetic Resonance (HNMR)

For select polymers, a portion of the purified solids was dissolved in an appropriate dueterated solvent (either deuterium oxide, Deuterated chloroform, or Deuterated methyl sulfoxide depending on solubility). The solution was placed in an HNMR glass tube and analyzed by HNMR at a minimum frequency of 300 MHz.

FTIR

Polymers which were soluble in dichloromethane were dissolved in DCM and cast coated onto salt plates. Polymers that were not soluble in dichloromethane were ground to a fine powder, mixed with potassium bromide and compressed into a pellet. The samples were measured using a Thermo Mattson, Satellite FTIR, Model 960M0017 with standard sample holder. This instrument was operated and data analyzed utilizing WinFirst software. Each run was performed by doing 35 scans from 400 cm⁻¹ to 4000 cm⁻¹ in transmittance mode at a resolution of 4.0 and a gain of 1. The mirror, processing, and signal settings were all at factory default. Prior to loading samples a background scan was collected with the sample chamber empty. After a background scan was collected each sample was placed in the sample chamber in the laser path and scanned.

Cell Growth

Hydrogels were weighed into scintillation vials and sterilized using Anprolene AN74i ethylene oxide gas sterilizer for 12 hour cycle per manufacturer's instructions. Hydrogels were then dissolved in cell growth media which consisted of DMEM/F12+Glutamax™ basal media supplemented with 5% v/v fetal bovine serum, penicillin (100 units/mL), and streptomycin (100 g/mL). Appropriate amount of growth media was added to give desired % w/v concentration for each hydrogel. After adding cell growth media hydrogels were allowed to fully dissolve at refrigerated conditions (approximately 2-8° C.) for ˜48 hours.

MCF-7 cells were cultured in 75 cm² flasks until ˜70-90% confluence was reached. Cells were prepared by first removing media from culture flask, washing with DPBS (Dulbecco's Phosphate Buffered Saline) and then adding 0.025% trypsin in EDTA solution and allowing 3-5 minutes of incubation at 37° C./5% CO₂ to detach cells from the surface of the flask. Fresh media was then added to flask to neutralize trypsin activity and re-suspend cells. This cell suspension was then transferred to a 15 mL conical bottom tube and centrifuged at ˜200 g for three minutes to produce a cell pellet in the bottom of the tube. Media was then removed carefully as to not disturb the cell pellet. At this point, the cell pellet was re-suspended in fresh media or cold hydrogels dissolved in media.

Hydrogels were pipetted into flat bottom polystyrene multi-well tissue culture plates with low evaporation lid. These were then placed in VWR Symphony 3405 air jacketed incubator at 37° C./5% C02 for 90 minutes to firm hydrogels. Hydrogels were then seeded with MCF-7 cells by pipetting cells suspended in media on top of gels and rocking plates back and forth to disperse cell suspension. After seeding with MCF-7 cells, multi-well plates were placed in incubator at 37° C./5% CO₂. Experiments were also performed by suspending cell pellets in cold hydrogel/cell media mixtures using AO20 and AO25. These mixtures were then pipetted into multi-well plates and placed in incubator at 37° C./5% C02.

Procedures described above including dissolving hydrogels in growth media and seeding cells on hydrogels were carried out under aseptic conditions using Esco class II type A2 biological safety cabinet. After a period of incubation time ranging from twenty-four hours to seven days, photos were taken using Amscope IN300t-FL phase contrast microscope. During the time cells were incubated media was replaced approximately every 48-72 hours or as color change of media indicated drop in pH.

### Cell Growth Examples

### Example 1: Poly(NIPAM-Co-Acrylic Acid) (AO14)

After synthesis, the Example thermogel was characterized as described above with the following results:

Rheological

The viscosity of Example 1 for a 1% (w/v) solution was found to be 0.07059 Pa·s in water at 5° C. The rheological G′ for example 1% in water hit a maximum of 0.1 Pa at 45° C. The gelation properties of AO14 were separately found to be dependent on pH with a favoring of gelation at low pH values.

HNMR

The HNMR spectra of Example 1 indicates peaks at following locations and intensities in following format location ppm (intensity, description): 1.0 ppm (100.00, broad), 1.5 ppm (34.76, broad), 2 ppm (18.11, broad), 2.7 ppm (0.19, single), 3.6 ppm (0.11, single), 3.8 ppm (16.45, broad), 7.7 ppm (2.14, broad). These results are consistent with the indicated polymer indicating successful synthesis.

FTIR

The FTIR spectra indicated a broad peak at 3500-3300 cm⁻¹, a sharp triplet peak at 2900-2700 cm⁻¹, strong absorption peaks at 1700 cm⁻¹, 1500 cm⁻¹, as well as weaker peaks at 1400 cm⁻¹, 1300 cm⁻¹, and a broad peak at 1200-1100 cm⁻¹. These results are consistent with the indicated polymer indicating successful synthesis.

Cell Growth

Cell growth in AO14 1% (w/v) dissolved in cell growth media resulted in typical two dimensional cell morphology with a flat, trapezoidal shape. Cells adhered to surface of wells much like typical growth in culture flasks. This Example shows that not any thermogel can function to generate 3D spheroidal pattern.

CCD-1068SK Fibroblasts: Not tested.

HEP G2 Hepatic Cells: Not tested.

VERO African Green Monkey Kidney Cells: Not tested.

### Example 2. Poly(Pluronic F127-Urethane) (AO20)

After synthesis, the Example thermogel was characterized as described above with the following results:

Rheological

For rheological testing a 10% w/v solution was generated in cold water. This solution was first tested for viscosity which was found to be 0.1018 Pa·s at 5° C. Upon temperature ramping the maximum gel strength obtained was a G′ of 4000 at 45° C. with onset at 27.5° C. The G′ at 37° C. was 1500 Pa.

HNMR

HNMR spectra was collected from polymer dissolved in Deuterated water. The HNMR spectra of Example 2 indicates peaks at following locations and intensities in following format “location” ppm (intensity, description): 1.2 ppm (0.75, broad), 1.7 ppm (16.03, broad), 1.9 ppm (100.00, broad), 2.1 ppm (1.07, broad), 2.4 ppm (0.46, broad). These results are consistent with the indicated polymer indicating successful synthesis.

FTIR

The FTIR spectra indicated a broad peak at 3500-3300 cm⁻¹, a strong peak at 3000-2700 cm⁻¹, weak absorption peaks at 2600 cm⁻¹, 1900 cm⁻¹, 1700 cm⁻¹, 1500 cm⁻¹, 1400 cm⁻¹, 1300 cm⁻¹, and 900 cm⁻¹ as well as a very strong broad peak at 1200-1000 cm⁻¹. These results are consistent with the indicated polymer indicating successful synthesis.

Cell Growth

MCF-7 Breast Cancer:

Cell growth in AO20 10% (w/v) dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Cellular growth was observed four days after seeding cells on AO20 hydrogel that has been thermally gelled before adding cell suspension on top. Cell growth was also observed seven days after cells suspended in cold hydrogel Spheroids reach a larger size when seeded on warmed hydrogel versus those suspended in cold hydrogel. Spheroids in warm hydrogel range in size from 60-120 μm with most being ˜100 μm. Spheroids grown by mixing in initially liquid hydrogel range in size from 20-60 μm with a fairly even size distribution. Level of aggregation 0 after four days of growth.

CCD-1068SK Fibroblasts:

Cell growth in AO20 10% (w/v) dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Cells showed spheroid formation when seeding on AO20 hydrogel that has been thermally gelled before adding cell suspension on top as well as cells suspended in cold hydrogel. Spheroids ranged in size from 40-180 μm with most being ˜80 μm. Level of aggregation 0 after four days of growth.

HEP G2 Hepatic Cells:

Cell growth in AO20 10% (w/v) dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Cells showed spheroid formation when seeding on AO20 hydrogel that has been thermally gelled before adding cell suspension on top as well as cells suspended in cold hydrogel. Spheroids ranged in size from 60-160 μm with most being ˜80 μm. Level of aggregation 2 after four days of growth.

VERO African Green Monkey Kidney Cells:

Cell growth in AO20 10% (w/v) dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Cells showed spheroid formation when seeding on AO20 hydrogel that has been thermally gelled before adding cell suspension on top as well as cells suspended in cold hydrogel. Spheroids ranged in size from 40-200 μm with even size distribution. Two dimensional attached growth also occurred simultaneously in some wells. Level of aggregation 1 after four days of growth.

### Example 3. Stearate Modified Methylcellulose (AO25)

After synthesis, the Example thermogel was characterized as described above with the following results:

Rheological

Example 3 was dissolved 5% w/v in distilled water and tested by rheology. The viscosity of the 5% w/v solution at 5 C was 0.1742 Pa·s. The maximum G′ reached was 80 Pa at 45° C. with onset at 35° C.

HNMR

The HNMR spectra of Example 3 indicates peaks at following locations and intensities in following format “location” ppm (intensity, description): 1.0 ppm (0.58, doublet), 2.7 ppm (44.30, broad), 3.0 ppm (8.70, broad), 3.3-4.0 ppm (100.00, broad), 4.7 ppm (9.33, broad). These results are consistent with the indicated polymer indicating successful synthesis.

FTIR

The FTIR spectra indicated a broad peak at 3600-3300 cm⁻¹, a weak peak at 2900 cm⁻¹, a strong peak at 1200-1000 cm⁻¹, and a weak peak at 900 cm⁻¹. These results are consistent with the indicated polymer indicating successful synthesis.

Cell Growth

MCF-7 Breast Cancer:

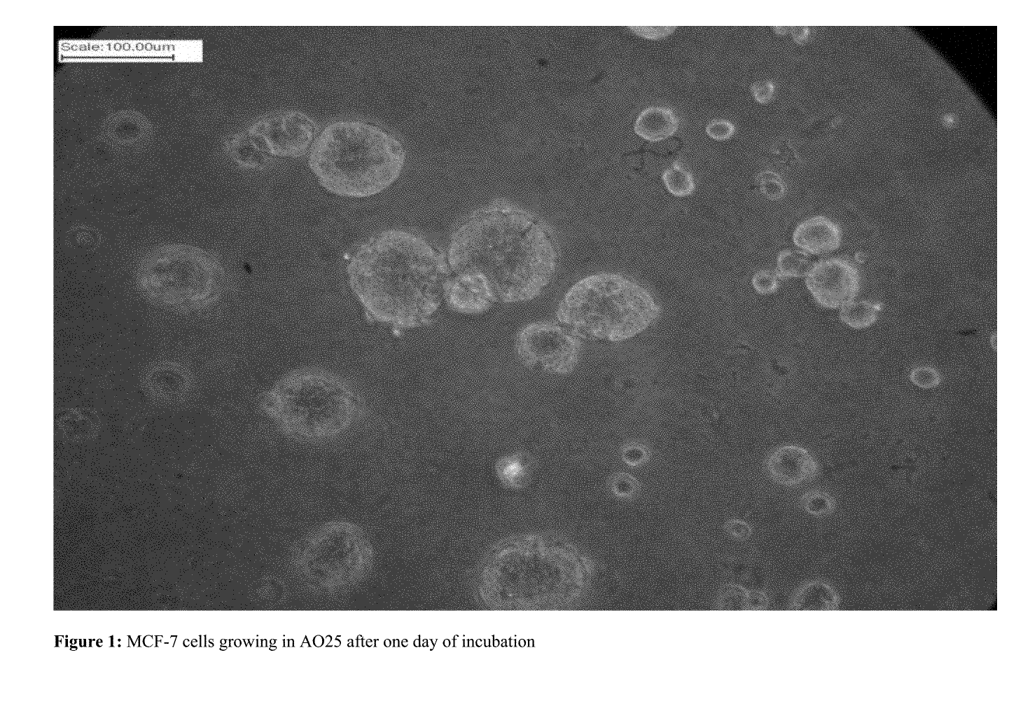

Cell growth in AO25 5% w/v dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Cell growth ˜24 hours after seeding cells on AO25 hydrogel that has been thermally gelled before adding cell suspension on top was observed. Cell growth seven days after cells suspended in cold hydrogel was also observed. Spheroids reach a larger size when seeded on warmed hydrogel versus those suspended in cold hydrogel. Spheroids grown on warmed hydrogel range in size from 30-110 m with most being larger than 50 μm. Spheroids grown by mixing in liquid hydrogel range in size from 20-60 μm with a fairly even size distribution. The image in FIG. 1 shows MCF-7 cells as described in Example 3 incubated for approximately 30 hours. The red arrow indicates an example of cells which have morphology indicating spheroidal growth. This is indicated by the overall spheroid morphology with fused cells that do not show distinguishable individual cell boundaries, i.e., without distinctive cellular membrane at cell-cell interfaces. Level of aggregation 0 after one day of growth and subsequently at four days of growth.

CCD-1068SK Fibroblasts:

Cell growth in AO25 5% w/v dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Spheroids reach a larger size when seeded on warmed hydrogel versus those suspended in cold hydrogel. For seeding on warmed hydrogel spheroids ranged in size from 40-180 μm with most being ˜100 μm. Seeding by suspending cells in cold hydrogel resulted in spheroids initially 20-60 μm but after extended incubation times (i.e. greater than 10 days) with media replacement spheroids increased in size to ˜100-120 μm. Level of aggregation 1 after four days of growth.

HEP G2 Hepatic Cells:

Cell growth in AO25 5% w/v dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Spheroids ranged in size from 60-140 μm with most being ˜80 μm. Additionally some irregular cell growth occurred in three dimensional sheets of cells suspended in gel. Level of aggregation 3 after five days of growth.

VERO African Green Monkey Kidney Cells:

Cell growth in AO25 5% w/v dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. Spheroids range in size from 20-120 μm with a most being ˜80 μm. Level of aggregation 2 after one day of growth and at four days of growth.

### Example 4. Poly(Poloxamer 407-Methylenediphenyldiisocianate) (AO26)

After synthesis, the Example thermogel was characterized as described above with the following results:

Rheological

The polymer was dissolved as a 5% w/v solution in cold water and tested by rheology. The viscosity of this solution at 5° C. was found to be 0.1801 Pa·s. The highest measured G′ was 440 Pa with gelation onset around 15° C. The G′ at 37° C. was 130 Pa.

HNMR

The HNMR spectra of Example 4 in CDCl3 indicates peaks at following locations and intensities in following format “location” ppm (intensity, description): 0.9 ppm (9.33, broad), 1.0 ppm (1.05, sharp), 1.2 ppm (18.80, broad), 1.3 ppm (9.47, broad), 1.7 ppm (5.86, multiple) 3.3 ppm (6.15, broad), 3.5 ppm (100.00, broad), 3.7 ppm (0.69, sharp), 3.9 ppm (1.82, broad), 4.3 ppm (0.33, broad), 5.2 ppm (0.06, sharp), 6.0 ppm (0.12, triplet), 7.0 ppm (0.81, broad), and 7.4 ppm (0.51, broad). These results are consistent with the indicated polymer indicating successful synthesis.

FTIR

The FTIR spectra indicated strong peaks at 3000-2700 cm⁻¹ and 1200 cm⁻¹ and medium peaks at 1450 cm⁻¹, 1350 cm⁻¹, 1300 cm⁻¹, 1250 cm⁻¹, 900 cm⁻¹ and 800 cm⁻¹. These results are consistent with the indicated polymer indicating successful synthesis.

Cell Growth

MCF-7 Breast Cancer:

Cell growth in AO26 5% w/v dissolved in cell growth media initially resulted in two dimensional cell morphology with a flat, trapezoidal shape but with somewhat rounded edges. Cells adhered to surface of wells much like typical growth in culture flasks. After a period of 5 days the same well in multi-well culture plate shows that cells have detached from the surface and formed spherical shaped cell colonies. These spherical growths appear to be contained in a narrow portion of the hydrogels just above the surface of the multi-well culture plate. Some of the growths are irregular in shape being more oblong than spherical. They range in size from 20-120 μm with most being ˜75 μm. Level of aggregation 1 after five days of growth.

CCD-1068SK Fibroblasts:

Cell growth in AO26 5% w/v dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. They range in size from 40-120 μm with most being ˜80 μm. Level of aggregation 1 after four days of growth.

HEP G2 Hepatic Cells:

Cell growth in AO26 5% w/v dissolved in cell growth media resulted in three dimensional cell morphology. Cells grew spheroids and thick sheets of cells suspended in hydrogel unlike typical growth in culture flasks. Spheroidal colonies of cells are suspended throughout the hydrogel at varying depths from surface of multi-well culture plate. Level of aggregation 3 after two days of growth.

VERO African Green Monkey Kidney Cells:

Cell growth in AO26 5% w/v dissolved in cell growth media resulted in three dimensional cell morphology. Cells grew spheroids suspended in hydrogel unlike typical growth in culture flasks. Spheroidal colonies of cells are suspended throughout the hydrogel at varying depths from surface of multi-well culture plate. They range in size from 40-140 μm with most being ˜80 μm. Some surface growth also occurred. Level of aggregation 2 after four days growth.

### Example 5. Poly(Vinylcaprolactam) (AO31)

After synthesis, the Example thermogel was characterized as described above with the following results.

Rheological

The example 5 polymer was dissolved as 20% w/v in cold distilled water. This was tested by rheology and the viscosity at 5° C. was found to be 0.07835 Pa·s. The maximum G′ obtained upon temperature ramp was 100,000 Pa at 45° C. with onset initiating at 30° C. The G′ at 37° C. was 60,000 Pa.

HNMR

The HNMR spectra was collected in D₂O. The HNMR spectra of Example 5 indicates peaks at following locations and intensities in following format “location” ppm (intensity, description): 0.9 ppm (3.33, split), 1.2-2.0 ppm (58.40, broad), 2.0-2.75 ppm (17.56, broad), 2.7-3.7 ppm (16.83, broad), 3.7-4.5 ppm (6.85, broad) 5.7 ppm (0.55, multiple). These results are consistent with the indicated polymer indicating successful synthesis.

FTIR

The FTIR spectra indicates strong peaks at 3500-3300 cm⁻¹, 3000-2700 cm⁻¹, 1700 cm⁻¹, 1400 cm⁻¹ and medium peaks at 2300 cm⁻¹, 1300-1000 cm⁻¹, 900 cm⁻¹, 800 cm⁻¹, 700 cm⁻¹ and 600 cm⁻¹. These results are consistent with the indicated polymer indicating successful synthesis.

Cell Growth

MCF-7 Breast Cancer:

Cell growth in AO31 20% (w/v) dissolved in cell growth media initially resulted in typical two dimensional cell morphology with a flat, trapezoidal shape. Cells adhered to surface of wells much like typical growth in culture flasks. Cells would rapidly die and detach from surface and appear as sheets of cells floating in media. Similar to Example 1, this Example shows that only specific thermogelling polymers work.

CCD-1068SK Fibroblasts: Not tested.

HEP G2 Hepatic Cells: Not tested.

VERO African Green Monkey Kidney Cells: Not tested.

Discussion

Of the polymers tested, there are two important properties in regards to the polymer having the capacity to serve as a spheroid-activating growth media.

First, the polymer must have the capacity to form into a thermogel at 37° C. which has suitable stiffness and viscosity to mimic the mechanical properties which normally encompass cancerous cells in vivo. Example 1 shows a weakly thermogelling polymer in which the cells simply grow in a traditional 2D format along the bottom of the well.

Second, the polymer must have suitable biocompatibility to allow for cell attachment and growth. As shown in Example 5, the polymer is not conducive to cell growth despite being a thermogel. Cellular extracts, e.g., Matrigel, or synthetic poly(amino acids) have been used as a matrix to grow 3D cell spheroids, but this is the first time to show that synthetic and semi-synthetic polymers which do not have amino acids display the property of promoting 3D cell spheroid formation.

These new synthetic and semi-synthetic thermogelling polymers allow for growing tumor cells in a media which is conducive towards generating bio-relevant tumor morphology in a reproducible manner.

It will be appreciated that the Examples presented herein are meant to further describe the invention, and not to limit the scope thereof.

What is claimed is:

1. A platform for inducing 3D spheroid formation of biological cells, comprising: a thermogelling polymer without amino acids.

2. The platform for inducing 3D spheroid formation of biological cells of claim 1, wherein the thermogelling polymer without amino acids is a stearate-modified methyl cellulose.

3. The platform for inducing 3D spheroid formation of biological cells of claim 1, wherein the thermogelling polymer without amino acids is poloxamer 407 linked by polyurethane linkages.

4. The platform for inducing 3D spheroid formation of biological cells of claim 3, wherein the polyurethane linkages are generated utilizing hexamethyldiisocyanate.

5. The platform for inducing 3D spheroid formation of biological cells of claim 3, wherein the polyurethane linkages are generated utilizing methylene diphenyldiisocyanate.

6. The platform for inducing 3D spheroid formation of biological cells of claim 3, wherein the polyurethane linkages are generated utilizing toluene diisocyanate.

7. The platform for inducing 3D spheroid formation of biological cells of claim 1, wherein the thermogelling polymer without amino acids is a cellulose derivative modified by chemical conjugation of stearic acid units.

8. The platform for inducing 3D spheroid formation of biological cells of claim 1, wherein the thermogelling polymer without amino acids is a cellulose derivative modified by chemical conjugation of methyl units.

9. The platform for inducing 3D spheroid formation of biological cells of claim 1, wherein the thermogelling polymer without amino acids is a cellulose derivative modified by chemical conjugation of hydroxypropyl units.

10. The platform for inducing 3D spheroid formation of biological cells of claim 1, wherein the thermogelling polymer without amino acids is a cellulose derivative modified by chemical conjugation of ethyl units.

11. The platform for inducing 3D spheroid formation of biological cells of claim 1, wherein the thermogelling polymer without amino acids is a cellulose derivative modified by chemical conjugation of propyl units.

12. The platform for inducing 3D spheroid formation of biological cells of claim 1, wherein the thermogelling polymer without amino acids is a cellulose derivative modified by chemical conjugation of hydroxyethyl units.

13. The platform for inducing 3D spheroid formation of biological cells of claim 1, wherein the thermogelling polymer without amino acids is a cellulose derivative modified by chemical conjugation of carboxymethyl units.

14. The platform for inducing 3D spheroid formation of biological cells of claim 1, further comprising a cell growth medium in the range of about 1 to about 30% w/v.

15. The platform for inducing 3D spheroid formation of biological cells of claim 14, wherein the cell growth medium is in the range of about 5 to about 20% w/v.

16. The platform for inducing 3D spheroid formation of biological cells of claim 1, wherein the biological cells are tumor cells.

17. The platform for inducing 3D spheroid formation of biological cells of claim 1, wherein the biological cells are benign tumor cells.

18. The platform for inducing 3D spheroid formation of biological cells of claim 1, wherein the biological cells are malignant tumor cells.

19. The platform for inducing 3D spheroid formation of biological cells of claim 1, wherein the biological cells are cancer cells.

20. The platform for inducing 3D spheroid formation of biological cells of claim 1, wherein the biological cells are breast cancer cells.

21. The platform for inducing 3D spheroid formation of biological cells of claim 1, wherein the spheroids have diameters of about 10 μm to about 1000 μm.

22. The platform for inducing 3D spheroid formation of biological cells of claim 1, wherein the spheroids have diameters of about 20 μm to about 200 μm.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)
